(19) United States
(12) Patent Application Publication
(10) Pub. No.: US 2011/0033563 A1
(43) Pub. Date: Feb. 10, 2011

(54) Stabilized Senna Extract Gel Formulation and Method of Preparation

(76) Inventor: Horacio Peraino, Sanchez de Bustamante (AR)

Correspondence Address: SARFARAZ K. NIAZI, 20 RIVERSIDE DRIVE, DEERFIELD, IL, 60015, US

(21) Appl. No.: 12/536,108

(22) Filed: Aug. 5, 2009

(51) Int. Cl. A61K 36/48; A61K 31/70

(52) U.S. Cl. 424/757; 514/23

(57) ABSTRACT

A stable formulation of sennosides with increased shelf-life and solubility comprising a granulate containing senna extract concentrate and, for each part by weight of senna extract concentrate, one to three parts by weight of Eudragit polymer.

The present invention relates to a stable form of senna extract comprising a granulate containing senna extract concentrate and, for each part by weight of senna extract concentrate, 0.1 to 0.3 parts of weight of Eudragit polymer in a gel formulation.

### BACKGROUND OF THE INVENTION

Vegetables extracts based on compounds with phenolic or polyphenols functional groups such as sennosides are not stable in aqueous solutions in the presence of oxygen. The polymers, which are formed cause turbidity and precipitation and result in the loss of pharmaceutically active material and the efficacy of the product.

In order to avoid these chemical instabilities, attempts have been made in the past that included formulations in the solid form such as Senokot (sugar coated tablets) from Purdue. Pursennid (sugar coated tablets) from Sandoz, Agiolax (granulate) from Maduus (in Germany)| or in semisolid form Tamarine (jam) from Serono. In the semisolid form (jam), the sennosides are present in acid form and not as calcium salts. In the acid form, sennosides are not soluble in water and therefore are protected from the degradation process.

The degradation process of sennosides in aqueous solution is slow but directly affected by the storage temperature. The degradation route of sennosides involves a preliminary breakdown of glucosidic bonds and, soon thereafter, oxidation and polymerization of dianthrones or from hydrolysis.

On the basis of pharmacological research on senna extract, it has been confirmed that the specific influence on colon motility (laxative effect) of sennosides must be ascribed to the anthrones or dianthrones and not to their degradation products (oxidized or polymerized products), which are not active. A reduction of pharmacological activity is expected when the products degrade. As a result, most products on the market today provide a shorter shelf life, particularly if this is a liquid product where the chances of degradation are much higher.

| | | | Time | | | |
|---|---|---|---|---|---|---|
| Test | | Limit | 6 months | 12 months | 18 months | 24 months |
| Apperance | Brown Jelly | Brown jelly | Unmodified | Unmodified | Unmodified | Unmodified |
| Taste | Sweet | Sweet | Unmodified | Unmodified | Unmodified | Unmodified |
| Odor | Characteristic | Characteristic | Unmodified | Unmodified | Unmodified | Unmodified |
| % Sugar | 70 | 70 | Unmodified | Unmodified | Unmodified | Unmodified |
| pH | 3.0-4.0 | 3.5 | Unmodified | Unmodified | Unmodified | Unmodified |
| Sennosides | 170 mg ± 10% | 166.0 mg | Unmodified | Unmodified | Unmodified | Unmodified |
| (per 100 g) | | | | | | |

There is therefore a need to invent a formulation that would be easily administered to most patients and thus requiring a non-solid dosage form, be stable for a longer time and have a pleasant taste and palatability.

### SUMMARY OF THE INVENTION

The object of the present invention is to provide a stable formulation of sennosides with extended shelf life. A further object is to provide such a formulation which has good taste and which is easy to use. These objects are achieved in a granulate containing senna extract concentrate and, for each part by weight of senna extract concentrate, 0.1 to 0.3 parts of weight of Eudragit resin in a jelly form.

Table 1. Stability Data on New Formulation Lot D-122 Jelly

From data obtained in stability studies it has been found that the active principles (sennosides) in the granulate are stable for at least 3 years compared to the maximum stability shelf life of 3 months at room temperature and 6 months in the refrigerator.

### DETAILED DESCRIPTION OF PREFERRED EMBODIMENTS

The method of preparation of instant invention consists of two distinctive steps. In the first step, senna extract granulate is manufactured, which is then incorporated in a jelly-type formulation for final administration to humans and animals.

Step 1: Preparation of Senna Extract Granulate. The bill of materials to prepare 20 kgs of senna extract granulate is provided in Table 2:

TABLE 2
Bill of Materials for Senna Extract Granulate

| Name | Quantity |
|---|---|
| Talc | 0.12 kg |
| Polyethylene Glycol 400 | 0.060 kg |
| Senna Extract 20% | 14.88 kg |
| Eudragit-L100 | 2.98 kg |
| Eudragit-L30 D55 | 1.96 kg |
| Isopropyl alcohol | 8.44 kg |
| Purified Water | 1.37 kg |

The method of manufacture of senna extract granulate comprises following steps:
1. In a 20 L stainless steel container, disperse with constant stirring and dissolve Eudragit L100 in isopropyl alcohol. Once it is homogenized, keep agitating rapidly and add Purified Water slowly forming a thread in the center of the stirring, until an emulsion, transparent, free from lumps is formed. Adding water quickly will cause precipitation; avoid it.
2. Divide the emulsion from step 1 into two equal parts by weight.
3. Add senna extract in a mixer and one-half of the emulsion from step 2 slowly.
4. Knead until the point of granulation, obtaining an amorphous mass that does not yield either liquid or powder and is consistent.
5. Place the mixture obtained from the step 3 in the drying oven in which the mixture should be placed well spread thin layers and shaken periodically to achieve faster drying.
6. Dry the mixture in the oven at 45° C. for approximately 15 hours or until the residual moisture are less than 1%.
7. Transfer dried granules from step 5 to a mixer and add slowly the balance of the emulsion from step 2 and granulate again, follow the steps 4-6 to obtain granules with residual moisture less than 7%.
8. Pass granules from step through mesh #16 and store until further use.
9. In a 20 L stainless steel container, disperse Talc and Purified Water (3.32 kg) with constant stirring form homogenous cakes; this may require stirring for about 30 minutes.
10. In a 20 L stainless steel container place through a sieve #40 mesh the solution of Eudragit-L30 D55, to eliminating waste of raw materials, when this process is complete, incorporate the Polyethylene glycol, and homogenize.
11. Add step 10 into step 9 and stir for about one hour until the mixture is fully homogeneous and keep stirred until applied to granules.
12. Load granules from step 8 in a coating pan, preheated to 55° C. and air pressure between 0.4 and 0.2 Mpa for 10 minutes with step 11.
13. Upon application of solution in step 12, allow coated granules to roll for about 10 minutes with heat turned off.
14. Sieve the coated granules through #12 mesh size, store in sealed polyethylene bag for further use. [These are now labeled as SENNA PTX01]

Step 2: Jelly Formulation. The senna extract granulate manufactured in step 1 is now formulated in a jelly dosage form. The bill of materials for the manufacture of 500 kg of the jelly dosage form is listed as Table 3

TABLE 3
Bill of Materials for Jelly Formulation

| Raw material | Quantity |
|---|---|
| Citric acid | 5.00 kg |
| Sucrose | 170.00 kg |
| Sodium benzoate | 0.35 kg |
| Prunes | 22.60 kg |
| Honey | 9.55 kg |
| Raisins | 191.70 kg |
| Flax seed | 12.50 kg |
| Potassium sorbate | 0.35 kg |
| Caramel | 4.63 kg |
| Purified water | 5.00 kg |
| Purified water | 5.00 kg |
| Purified water | 5.00 kg |
| Purified water | 60.00 kg |
| Senna Extract PTX01 | 8.33 kg |

1. In a stainless steel reactor heal purified water (60.00 kg) to a temperature of 40° C., adding under constant stirring sucrose (170.00 kg) and honey (9.55 kg), keep stirring.
2. Prepare the citric acid solution (5.00 kg citric acid in 5.00 Kg purified water) in a 20 L stainless steel container with constant stirring.
3. Add step 2 into step 1.
4. In a 20 L stainless steel container, prepare a sodium benzoate solution 0.35 kg sodium benzoate in 5.00 kg purified water.
5. Add step 3 into step 4.
6. In a 20 L stainless steel container, prepare a potassium sorbate solution (potassium sorbate 0.35 Kg in 5.00 kg purified water).
7. Add step 5 into step 6.
8. In a Butcher Boy grinder Machine with 3.5 mm diameter hole screen, grind prunes and then raisins, receiving milling in sanitized stainless steel trays.
9. While moving the reactor blades add to step 8, linnum seed (12.5 kg), and caramel (4.63 kg).
10. While keeping the reactor in agitation, incorporate senna extract PTX01, mixing for 30 minutes.
11. Keep stirring the reactor and increase the temperature between 55 to 60° C., hold for 20 minutes and transfer to storage tanks for packaging.
12. Final product specification: Appearance: viscous paste, dark brown, with characteristic odor and flavor. Flax seeds and small pieces of fruit can be observed; pH: 3.3-3.7 (dispersion in water 10% w/v), sucrose 68-73%. consistency: 3.3 to 3.9 cm

What is claimed is:

1. A granulate consisting essentially of senna extract, for each part by weight of senna extract concentrate, 1 to 3 parts of weight of Eudragit polymer.

2. A gel formulation comprising said granulate of senna extract wherein the proportion of active sennosides comprises 0.1 to 0.3% of the final product.

3. A method of treating diseases which respond to senna extract, which comprises administering an effective amount of the said granulate, set forth in claim 1.

5. A combination consisting essentially of (a) a granulate consisting of senna extract concentrate and, for each part by weight of senna extract concentrate, 1 to 3 parts of weight of Eudragit polymer.
